(19) United States
(12) Patent Application Publication
(10) Pub. No.: US 2002/0111642 A1
(43) Pub. Date: Aug. 15, 2002

(54) Adjustable saphenous vein clamp

(76) Inventors: John T.M. Wright, Denver, CO (US); David W. Skinkle, Denver, CO (US)

Correspondence Address: THOMAS D. BRATSCHUN, SWANSON & BRATSCHUN, LLC, 1745 SHEA CENTER DRIVE, SUITE 330, HIGHLANDS RANCH, CO, 80129, US

(21) Appl. No.: 09/784,518

(22) Filed: Feb. 15, 2001

(51) Int. Cl. A61B 17/08

(52) U.S. Cl. 606/158

(57) ABSTRACT

A clamp configured and constructed for use in clamping a saphenous vein for use in coronary bypass surgery comprising a pair of pivotally connected clamping members, each constructed and configured to define a vein engaging clamping structure and a handle element for actuating the clamp means pivotally connecting the clamping members biasing means for biasing the clamping structures toward each other and ratchet means for adjusting the biasing element to apply a variable desired clamping force to the clamping structures is disclosed.

### FIELD OF THE INVENTION

This invention relates to surgery and, specifically, to the transplantation of blood vessel, with particular advantageous use where space is limited. The most common use of this invention is expected to be in coronary artery bypass surgery wherein a section of the patient's saphenous vein is attached to the heart to provide blood flow and bypass a defective coronary artery. The invention may, however, be used to clamp any natural or synthetic blood vessel.

### BACKGROUND OF THE INVENTION

The use of the clamp in coronary bypass surgery is an exemplary use and is so described without intending to limit the scope of this patent to such uses.

In coronary bypass surgery a section of the patient's saphenous vein is removed from the leg and prepared to be implanted adjacent the patient's heart. One end of the vein implant is attached to the heart in fluid communication with the coronary artery distal from a blockage or other defect in the coronary artery. The other end of the vein implant is attached the aorta, or any source of aortic blood, thus providing a new source of blood to the heart.

Regardless of which attachment is made first there may be blood flow through the attached vein implant into the surgical cavity. It is important to be able to clamp the vein implant to prevent blood flow through it.

Prior art clamps used for this purpose are generally constructed to apply a fixed clamping force which may be insufficient to clamp off the vein implant or it may apply a to great a clamping force and damage the tissue of the vein implant.

The prior art clamps are, generally, “alligator” type clamps without means for adjusting the clamping force. Examples of such prior art clamps are the Applied Stealth and the Baxter SoftJaw clamps described in literature published by Applied Medical, Rancho Santa Margarita, Calif. Clamps of a similar design are described byt Dieter von Zeppelin in U.S. Pat. Nos. 4,971,055 and 5,074,870.

Another type of damp is depicted in U.S. Pat. No. 5,571,121 to Heifetz in which trauma is sought to be reduced by placing stops in the clamp, but this design limits the opening in the clamp but not the clamping force.

The object of this invention is to provide an adjustable force clamp for clamping a saphenous or other vein or blood vessel wherein the surgeon can adjust the clamping force easily and quickly during surgery.

### SUMMARY OF THE INVENTION

The present invention is a clamp configured and constructed for use in clamping a vein, such as a saphenous vein, that is used in bypass surgery or for clamping any other blood vessel to prevent the flow of blood there through. The clamp comprises a pair of pivotally connected clamping members, each having a vein engaging clamping element and a handle element for actuating the clamp, a biasing element, such as a torsion spring, for biasing the clamping elements together and means for adjusting the biasing element to apply any desired amount of force to the clamping elements.

### DESCRIPTION OF THE DRAWING

FIG. 1 is a generally isometric perspective view of the adjustable clamp of this invention.

FIG. 2 is side view of the clamp taken from the left as depicted in FIG. 1, illustrating in dashed lines elements of the adjusting mechanism of the clamp.

FIG. 3 is a plan view or the clamp taken from the bottom as depicted in FIG. 1.

FIG. 4 is a side view of the clamp taken from the right as depicted in FIG. 1 with the clamp inverted, depicting the clamping elements and actuating handles in the upper portion and the adjusting mechanism in the lower portion of the drawing.

FIG. 5 is a cross-sectional view of a portion of the adjusting mechanism taken along lines 5-5 in the direction of the arrows as depicted in FIG. 1.

FIG. 6 is an exploded view showing the elements of the present invention.

### DESCRIPTION OF THE PREFERRED EMBODIMENT

The embodiment shown and described hereinafter is the presently contemplated best mode of construction and is exemplary of the invention. The invention may, however, take on many configurations without departing from the concept of the invention and the scope of the claims. Any of many materials may, likewise, be used within the scope and content of the invention.

The clamp, a saphenous vein clamp in the preferred embodiment, comprises clamping members 10 and 20, adjusting ratchet knob 30, rachet plate 40 and bias spring 50.

Clamping member 10 comprises base plate 11 from which pivot post 12, having a central passage, extends upwardly and also from which rotation stop boss 13 extends upwardly a shorter distance. Clamping structure 14 extends downwardly on one extremity of the plate 11 and actuating grip 15 extends downwardly from a generally opposed extremity of the plate 11. This member is preferably composed of a biocompatible polymer and formed by injection molding.

Clamping member 20, also preferably composed of a biocompatible polymer and formed by injection molding, comprises base plate 21 from which guide cylinder 22, having a central passage configured and dimensioned to rotatably receive pivot post 12, and spring attaching boss 23, which has an aperture 23a, extend upwardly and from which clamping structure 24 extends downwardly on one extremity of the plate 21 and actuating grip 25 extends downwardly from a generally opposed extremity of the plate 21.

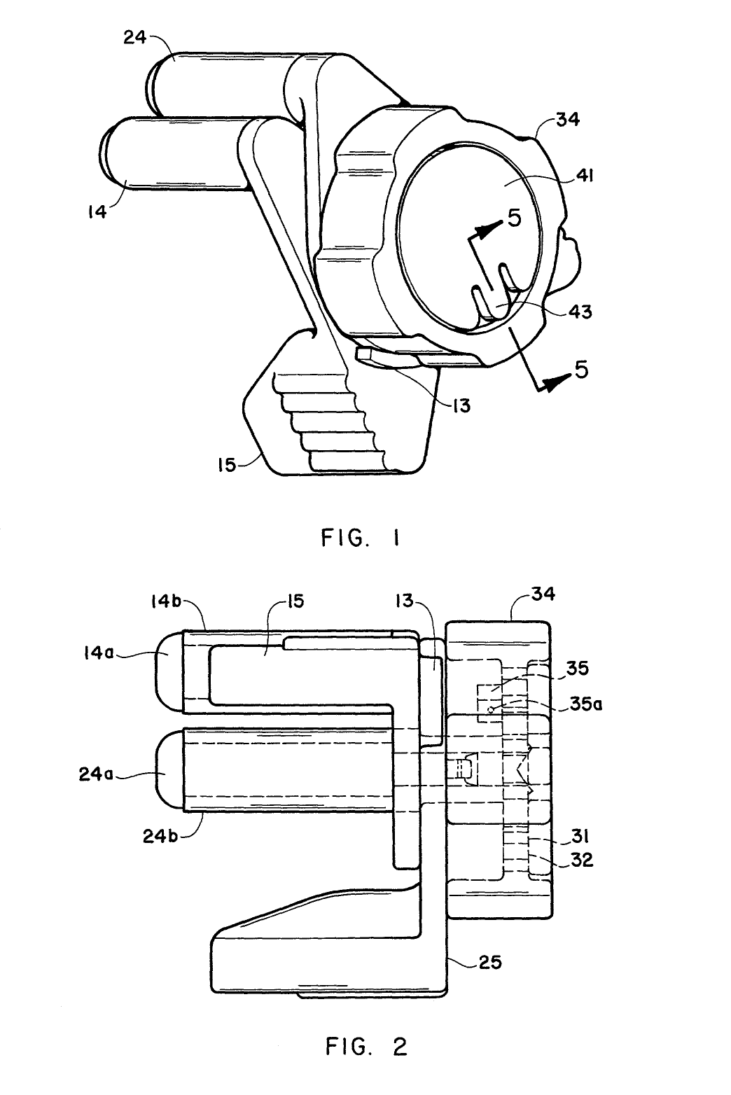

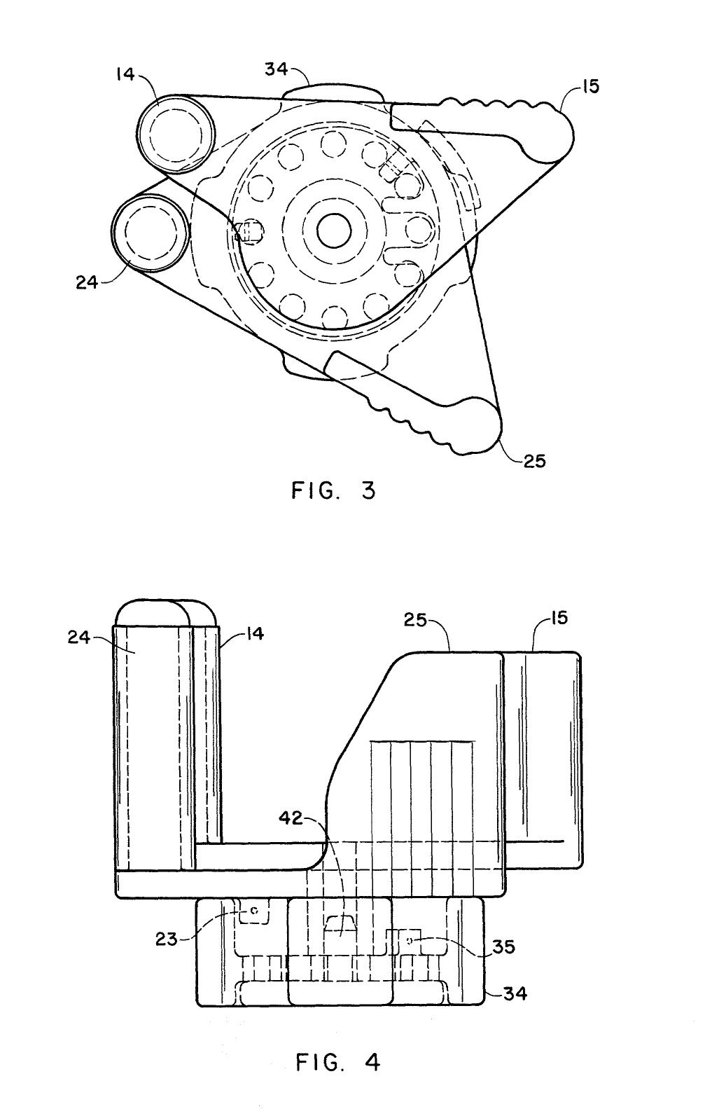

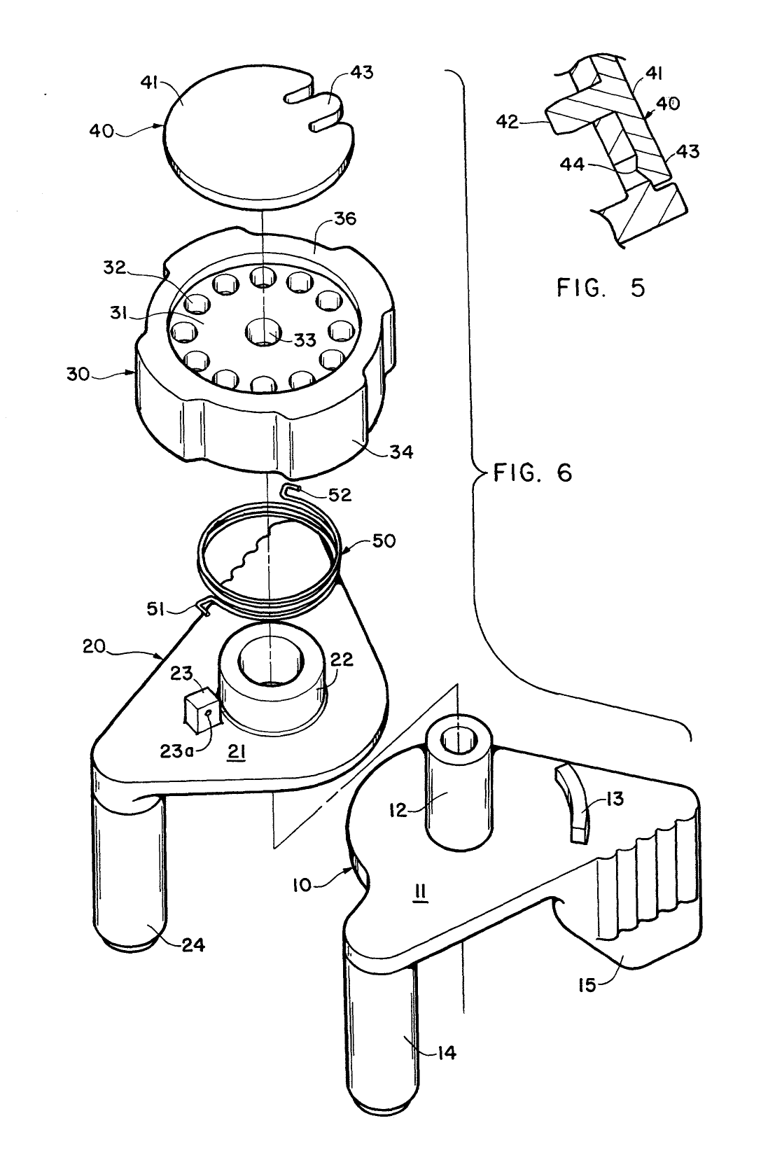

As best shown in FIG. 2, the clamping structures 14 and 24, respectively, comprise a post 14a and a soft resilient sleeve 14b, and a post 24a and a soft resilient sleeve 24b, to apply to the saphenous vein sufficient force to prevent blood flow there through with minimum trauma to the vein tissue.

Adjusting ratchet knob 30 comprises a central planar guide and rachet structure 31 through which a plurality of ratchet apertures 32 and a central pivot aperture 33 extend. Actuating knobs 34 are formed on the outer cylindrical periphery and an annular peripheral rim 36 extends upwardly. Spring connector boss 35 having aperture 35 extends downwardly from the periphery of the planar guide and rachet structure 31.

Rachet cap 40 comprises a ratchet member 41 the comprises a downwardly extending guide and attachment pin 42 and ratchet tongue 43 from which a ratchet stop 44 extends downwardly. Members 30 and 40 are also preferably composed of a biocompatible polymer and formed by injection molding.

The bias spring 50 is depicted in the exemplary drawing as a circular torsion spring encircling the guide cylinder 22 having an end 51 attached to the boss 23 through aperture 23a and an end 52 attached to the aperture 35a in boss 35 on the adjusting ratchet knob 30. A flat watch type torsion spring with suitable end hooks on the ends encircling all or a major part of the guide cylinder 22 is also contemplated as one of the preferred types of biasing means. Any spring that encircles at least a major portion, i.e. more than about half the circumference, of the guide cylinder 22 may be used.

The downwardly extending guide and attachment pin 42 is adhesively bonded in the central cylindrical portion of pivot post 12 that extends upwardly from the base plate 11 of clamping member 10.

The bias spring ends 51 and 52 are, respectively, engaged in the aperture 23a in attachment boss 23 on clamping member 20 and in aperture 35a in attachment boss 35 on adjusting knob 30. The torsional bias force of the spring is, thus, applied in one direction directly to the clamping member 20 and in the other direction to clamping member 10 through the ratchet structure comprising the rachet apertures 32 and rachet member 43 and the pivot post 12 to which the rachet cap 40 is adhesively bonded.

The magnitude of the clamping force between the clamping structures 14 and 24 is adjustable to enable the surgeon to assure the application of sufficient force to prevent blood flow there through with minimum trauma to the vein tissue by simply turning the ratchet knob 30 thereby changing the bias applied by the spring 50. The amount of force applied to the clamping structure is, as is apparent from the foregoing, a function of the position of the ratchet point 43-44 relative to the adjustment knob 30.

Various types of biasing springs may be used, of course, but a circular torsion spring is presently preferred. The configuration of the clamping members may be varied, and the configuration of the clamp handles is simply a matter of design choice.

### INDUSTRIAL APPLICATION

The invention is useful in the surgical instrument industry.

What is claimed is:

1. A clamp configured and constructed for use in clamping a saphenous vein for use in coronary bypass surgery comprising:
a pair of pivotally connected clamping members, each constructed and configured to define a vein engaging clamping structure and a handle element for actuating the clamp,
means pivotally connecting the clamping members;
biasing means for biasing the clamping structures toward each other; and
ratchet means for adjusting the biasing element to apply a variable desired clamping force to the clamping structures.

2. The invention of claim 1 wherein the biasing means comprises a generally circular torsion spring surrounding the means pivotally connecting the clamping members, one end of the torsion spring being connected to a clamping member and the other end of the torsion spring being clamped to the means for adjusting the biasing element to apply a variable desired clamping force to the clamping structures.

3. An adjustable saphenous vein clamp comprising: first clamping member (10-15) and second clamping member (20-25), an adjusting ratchet knob (30-36), ratchet means (40-44) and biasing means (50-52),
the first clamping member comprising a base plate (11), a pivot post (12) extending in a first direction from the base plate, and clamping structure (14) extending from the base plate in a second direction substantially opposite said first direction, and handle means (15);
the second clamping member comprising a base plate (21), spring attachment means (23) and guide means (22)constructed to define a central passage configured and dimensioned to rotatably receive pivot post of the first clamping member and clamping structure (24) extending from the base plate in said second direction, and handle means (25);
the ratchet knob being configured and constructed to define means engaging the ratchet means (44), and spring attachment means (35);
the ratchet means (40) comprising ratchet structure (44) and attachment means (42) attached to the pivot post on the first clamping member; and
the biasing means(50) being a spring having a first end (51) attached to spring attachment means on the second clamping member and a second end (52) attached to the spring attachment means on adjusting knob.

4. An adjustable blood vessel clamp comprising: a first clamping member and a second clamping member, a ratchet assembly and biasing means, the first and second clamping members each respectively defining opposed clamping structure for engaging and applying clamping pressure to a blood vessel and interacting pivoting structure to permit relative pivotal movement of said first and second clamping members, one of said clamping members being constructed to define spring attachment means, said ratchet structure comprising a ratchet gear and ratchet gear engaging means, one of said ratchet gear and ratchet gear engaging means being configured and constructed to define spring attachment means, the biasing means being a spring having first and second ends attached respectively to the respective spring attachment means, the aforesaid structure being so configured and constructed that movement of one of said ratchet gear and ratchet gear engaging means relative to the other of the ratchet gear and ratchet gear engaging means changes the force applied to the opposed clamping structures, the entire structure being configured, dimensioned and constructed and adapted to be actuated manually to apply adjustable clamping force to a patients blood vessel during surgery.

5. A clamp configured and constructed for use in clamping a saphenous vein for use in coronary bypass surgery comprising:
a pair of pivotally connected clamping members, each constructed and configured to define a vein engaging clamping structure and a handle element for actuating the clamp,
means including a guide member having a generally circular circumference pivotally connecting the clamping members;
means encircling at least a majority of the guide member for biasing the clamping structures toward each other; and
means for adjusting the biasing element to apply a variable desired clamping force to the clamping structures.
